(19) United States
(12) Patent Application Publication
(10) Pub. No.: US 2019/0079066 A1
(43) Pub. Date: Mar. 14, 2019

(54) METHOD FOR PREDICTING PHASE BEHAVIOR IN CHEMICAL ENHANCED OIL RECOVERY PROCESSES

(71) Applicant: Chevron U.S.A. Inc., San Ramon, CA (US)

(72) Inventors: Soumyadeep Ghosh, Houston, TX (US); Adwait Chawathe, Houston, TX (US); Sophany Thach, Houston, TX (US)

(73) Assignee: Chevron U.S.A. Inc., San Ramon, CA (US)

(21) Appl. No.: 16/128,129

(22) Filed: Sep. 11, 2018

Related U.S. Application Data
(60) Provisional application No. 62/557,029, filed on Sep. 11, 2017.

(51) Int. Cl. G01N 33/28; E21B 43/16; E21B 41/00

(52) U.S. Cl. CPC G01N 33/2823; E21B 41/0092; E21B 43/16

(57) ABSTRACT

A method for predicting phase behavior includes determining a hydrophilic-lipophilic difference based on a ratio of salinity to optimum salinity in the microemulsion system. The method further includes determining a mean solubilization ratio as a direct function of the hydrophilic-lipophilic difference at a same state as an optimum solubilization. The method also includes predicting phase behavior based on the determined mean solubilization ratio. The method additionally includes injecting a surfactant into a reservoir according to the predicted phase behavior.

### CROSS-REFERENCE TO RELATED APPLICATIONS

This application claims priority to U.S. provisional application No. 62/557,029 filed Sep. 11, 2017, which is incorporated by reference in its entirety herein.

### TECHNICAL FIELD

The present disclosure relates generally to a method for forecasting chemical enhanced oil recovery. In particular, the present disclosure relates to predicting phase behavior of a microemulsion system in a chemical enhanced oil recovery process using an average solubilization method. The present disclosure also relates generally to a method for designing a chemical solution for use in a chemical enhanced oil recovery process. The present disclosure further relates generally to a method of performing a chemical enhanced oil recovery process.

### BACKGROUND

In chemical enhanced oil recovery processes, a chemical solution (i.e., an injection fluid including at least one surfactant, co-surfactant, alkali, or co-solvent) is injected into an oil reservoir. For example, in surfactant flooding processes, surfactant mixes with the oil and water present in the reservoir forming microemulsion systems having one or more fluid phases. Various types of microemulsion systems may form having a microemulsion of oil, water (or brine), and surfactant. In particular, a single phase system, two-phase systems, or a three-phase system are possible. The single phase system includes a microemulsion phase. The two-phase system can include a microemulsion phase and an excess oil phase. Alternatively, the two-phase system can include a microemulsion phase and an excess brine phase. The three-phase system includes a microemulsion phase, an excess oil phase, and an excess brine phase.

The number of phases formed and the composition(s) of the phases vary depending upon variables associated with the microemulsion system, for example, the salinity, the surfactant formulation, temperature, and pressure.

It is desirable to model phase behavior in chemical enhanced oil recovery processes in order to perform reservoir simulations and forecast field scale oil recovery. Accurately predicting microemulsion phase behavior can shorten the laboratory screening process used in the design of chemical solutions (e.g., surfactant formulations) used in chemical enhanced oil recovery processes. The hydrophilic-lipophilic difference (HLD) correlation, developed by Salager et al. (Optimum formulation of surfactant/water/oil systems for minimum interfacial tension or phase behavior. Society of Petroleum Engineers Journal, 19(02), April 1979; and Partitioning of ethoxylated octylphenol surfactants in microemulsion-oil-water systems: Influence of temperature and relation between partitioning coefficient and physicochemical formulation. Langmuir, 16(13), pp. 5534-5539, 2000, doi: 10.1021/la9905517) (which are incorporated by reference in their entireties herein), has been widely accepted as a reliable starting point in designing surfactant formulations and has since been incorporated into various models for prediction of microemulsion phase behavior.

The Hydrophilic-Lipophilic Difference-Net Average Curvature (HLD-NAC) model for predicting phase behavior was first introduced by Acosta et al., Net-average curvature model for solubilization and supersolubilization in surfactant microemulsions. Langmuir, 19(1), 196-195 (2003) (which is incorporated by reference in its entirety herein). The underlying assumption of the NAC model is that the solubilized oil and water components in a microemulsion form domains (i.e., micelles) that are spherical in shape. Hence, domain size can be mathematically characterized by radius, or alternatively, by curvature. Curvature is the inverse of radius. Prediction of domain sizes (using either radius or curvature) in a microemulsion allows for the calculation of solubilization ratios, phase types, and phase volumes.

Subsequent models for predicting phase behavior have built upon the HLD-NAC model. These include the HLD-NAC with catastrophe theory model (Jin et al., Physics based HLD-NAC phase behavior model for surfactant/crude oil/brine systems. Journal of Petroleum Science and Engineering, 136, 68-77 (2015)) (which is incorporated by reference in its entirety herein), the HLD-NAC with surfactant continuum model (Nouraei et al., Predicting solubilization features of ternary phase diagrams of fully dilutable lecithin linker microemulsions. Journal of Colloid and Interface Science, 495, 178-190 (2017)) (which is incorporated by reference in its entirety herein), and modified HLD-NAC with characteristic length model (Ghosh et al., Dimensionless Equation of State to Predict Microemulsion Phase Behavior. Langmuir, 32(35), 8969-8979 (2016); Khorsandi et al., Robust Flash Calculation Algorithm for Microemulsion Phase Behavior. Journal of Surfactants and Detergents, 19(6) 1273-1287 (2016)) (which are incorporated by reference in their entireties herein). However, all of these HLD-NAC models have limitations with respect to the extent to which they accurately represent or predict real-world behavior. Accordingly, their predictive capabilities are limited.

Accordingly, there is a need for an improved method of predicting and modeling phase behavior in chemical enhanced oil recovery processes.

### SUMMARY

In accordance with the present disclosure, the above and other issues are addressed by the following:

In a first aspect, the present disclosure relates to a method for predicting phase behavior of a microemulsion system in a chemical enhanced oil recovery process. The method comprises determining a hydrophilic-lipophilic difference based on a ratio of salinity to optimum salinity in the microemulsion system. The method further comprises determining a mean solubilization ratio as a direct function of the hydrophilic-lipophilic difference at a same state as an optimum solubilization. The method also comprises predicting phase behavior based on the determined mean solubilization ratio. The method additionally comprises injecting a surfactant into a combination of oil and water at predetermined conditions and a predetermined amount to form fluid phases according to the predicted phase behavior.

In a second aspect, the present disclosure relates to a system for performing a chemical enhanced oil recovery process. The system comprises a computer interface configured to inject surfactant into a combination of oil and water to form an experimental microemulsion system. The system further comprises a processing unit configured to receive a data stream comprising experimental data from the experimental microemulsion system. The system also comprises a memory communicatively connected to the processing unit, the memory storing instructions which, when executed by the processing unit, cause the system to perform a method for predicting phase behavior in chemical enhanced oil recovery. This method for predicting phase behavior in chemical enhanced oil recovery comprises determining a hydrophilic-lipophilic difference based on a ratio of salinity to optimum salinity in the microemulsion system containing oil, water, and a surfactant; determining a mean solubilization ratio as a direct function of the hydrophilic-lipophilic difference at a same state as an optimum solubilization; and predicting phase behavior based on the determined mean solubilization ratio. Additionally, the system comprises a device for injecting a surfactant into a combination of oil and water at predetermined conditions and a predetermined amount to form fluid phases according to the predicted phase behavior.

In a third aspect, the present disclosure relates to a method of performing a chemical enhanced oil recovery process. The method comprises predicting phase behavior of a microemulsion system in an oil reservoir comprising a surfactant, oil, and water from an experimental microemulsion system comprising the same surfactant formulation. Predicting phase behavior involves determining a hydrophilic-lipophilic difference based on a ratio of salinity to optimum salinity in the experimental microemulsion system; determining a mean solubilization ratio as a direct function of the hydrophilic-lipophilic difference at a same state as an optimum solubilization; and predicting phase behavior based on the determined mean solubilization ratio. The method further comprises injecting the surfactant into the oil reservoir at predetermined conditions and a predetermined amount to form fluid phases according to the predicted phase behavior.

In another aspect, the present disclosure relates to a method for predicting phase behavior of a microemulsion system in a chemical enhanced oil recovery process. The method comprises determining a hydrophilic-lipophilic difference based on a ratio of salinity to optimum salinity in the microemulsion system. The method further comprises determining a mean solubilization ratio as a direct function of the hydrophilic-lipophilic difference. The method also comprises predicting phase behavior based on the determined mean solubilization ratio.

In yet another aspect, the present disclosure relates to a system for performing a chemical enhanced oil recovery process. The system comprises a processing unit configured to receive a data stream comprising experimental data from the experimental microemulsion system. The system also comprises a memory communicatively connected to the processing unit. The memory storing instructions which, when executed by the processing unit, cause the system to perform a method for predicting phase behavior in chemical enhanced oil recovery. This method for predicting phase behavior in chemical enhanced oil recovery comprises determining a hydrophilic-lipophilic difference based on a ratio of salinity to optimum salinity in the microemulsion system containing oil, water, and a surfactant. This method also comprises determining a mean solubilization ratio as a direct function of the hydrophilic-lipophilic difference at a same state as an optimum solubilization. This method further comprises predicting phase behavior based on the determined mean solubilization ratio.

In another aspect, the present disclosure relates to a method of performing a chemical enhanced oil recovery process. The method comprises predicting phase behavior of a microemulsion system in an oil reservoir comprising a surfactant, oil, and water from an experimental microemulsion system comprising the same surfactant formulation by: determining a hydrophilic-lipophilic difference based on a ratio of salinity to optimum salinity in the experimental microemulsion system; determining a mean solubilization ratio as a direct function of the hydrophilic-lipophilic difference at a same state as an optimum solubilization; and predicting phase behavior based on the determined mean solubilization ratio.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a flow chart illustrating steps to determine fitting parameters for surfactant solutions based on experimental data.

FIGS. 2-4 show exemplary symmetrical and asymmetrical functions of dimensionless average solubilization versus hydrophilic-lipophilic difference (HLD) scaled to optimum solubilization using different values of fitting parameters.

FIG. 5 depicts an exemplary fit of experimental data to the harmonic mean of the solubilization ratio/the optimum solubilization ratio as a function of the product of the optimum solubilization ratio and hydrophilic-lipophilic difference (HLD).

FIG. 6. shows inputs and outputs of a reservoir simulator for predicting phase behavior in chemical enhanced oil recovery processes disclosed herein.

FIG. 7 shows an exemplary ternary phase diagram resulting from the method for predicting phase behavior in chemical enhanced oil recovery processes disclosed herein.

FIG. 8 shows an exemplary ternary phase diagram resulting from the prior hydrophilic-lipophilic difference-net average curvature (HLD-NAC) model.

FIG. 9A shows an exemplary symmetric binodal curve for a mean solubilization ratio of 3 cc/cc.

FIG. 9B shows an exemplary symmetric binodal curve for a mean solubilization ratio of 10 cc/cc.

FIG. 10 depicts a computing system 102 configured to implement a method for predicting phase behavior in chemical enhanced oil recovery processes, which is prepared to inject a surfactant formulation into a combination of oil and water in an experimental setup 124 or an oil reservoir 126.

FIG. 11 depicts an exemplary system for performing a chemical enhanced oil recovery process.

FIG. 12 provides a flow chart illustrating steps in the method for predicting phase behavior in chemical enhanced oil recovery processes disclosed herein.

FIG. 13 shows examples of asymmetrical model outputs.

FIG. 14 shows predicted values compared to actual experimental data to show the validity of correlations described in Eqs. (19) and (20).

FIG. 15 shows correlations of optimum solubilization ratio (σ*) as a function of optimum salinity (S*). The 20 experiments with different oils at different temperatures were repeated 4 times (total 80). The goodness of fit of the correlation increases as the experimental data from repetitions are averaged (Right) with an R² of 0.97 as compared to R²=0.76 for all the 80 experiments (Left).

FIG. 16 shows the normalized average solubilization ratio as a function of the product of optimum solubilization ratio and HLD. The model fit is in good agreement with the experimental data.

FIG. 17 shows model prediction from tuned HLD-AST (hydrophilic-lipophilic difference-average solubilization theory) equation of state is in good agreement with actual experimental data.

FIGS. 18A-C show examples of tuned HLD-AST model outputs (solid lines) in comparison to experimental data (circles) for dodecane (EACN=12), pentadecane (EACN=15) and octadecane (EACN=18) at temperatures of 65° C. and 95° C. Blue represents σ₁ while red represents σ₂.

FIG. 19 shows model prediction from tuned HLD-AST equation of state compared to experimental data (left) with corresponding prediction of co-solvent concentrations (v/v) (right) in the microemulsion phase (dashed red) and the excess brine (dashed blue) phase. Data from Dwarakanath et al. (2008). Using Co-solvents to Provide Gradients and Improve Oil Recovery During Chemical Flooding in a Light Oil Reservoir. SPE Symposium on Improved Oil Recovery. Society of Petroleum Engineers (which is incorporated by reference in its entirety herein).

FIG. 20 shows an example of evolution of the three-phase region in the optimum salinity vs EACN space as a result of a constant model input of 0.4 and −0.6 for HLD_{U} and HLD_{L}. Equation (20) was used to calculate the optimum trend at a temperature of 65° C.

FIGS. 21A-B show examples of tuned HLD-AST model predictions as a function of HLD as typically seen in a salinity scan using model parameters listed in

Table 2. Green represents the type I region. Blue represents the type II region. Red represents the type III tie triangle. The region in the ternary space with no color represents the single-phase region (type IV microemulsion).

### DETAILED DESCRIPTION

Ghosh et al., “An Equation of State to Model Microemulsion Phase Behavior in Presence of Co-solvents Using Average Solubilization Theory”, SPE-191530-MS, SPE Annual Technical Conference and Exhibition, Dallas, Tex., September 2018 is incorporated by reference in its entirety herein.

In the following detailed description, reference is made to the accompanying drawings showing by way of illustration specific embodiments of methods and systems disclosed herein. It is to be understood that other embodiments may be utilized and logical changes may be made without departing from the scope of the present disclosure. The following detailed description, therefore, is not to be taken in a limiting sense.

The term “brine” as used herein refers to an aqueous solution of salts and other water soluble compounds. The term “water” is used interchangeably with the term “brine” herein.

The term “microemulsion system” as used herein refers to a microemulsion of oil and brine existing as solubilized domains with surfactant occupying the interface between the solubilized domains. The microemulsion system may include more than one surfactant, as well as, co-surfactant, alkali, and/or co-solvent.

The term “HLD” refers to hydrophilic-lipophilic difference. Hydrophilic-lipophilic difference is a function of the variables associated with the microemulsion system, for example, the salinity, the surfactant formulation, temperature, and pressure. HLD measures the deviation of a microemulsion system from the optimum state where oil and water are equally solubilized in a three-phase system. HLD is zero at optimum, positive when over-optimum, and negative when under-optimum. When only the salinity (S) is changing in the system with all other variables are fixed, the HLD relative to optimum salinity (S*) is expressed as:

$$HLD {\vert}_{T , P , EACN , f ( A ) , Cc} = ln ( \frac{S}{{S}^{*}} ) \qquad ( 1 )$$

Co-solvents are used with surfactants in modern chemical enhanced oil recovery (CEOR) formulations to avoid formation of viscous microemulsion phases (and reduce costs) in porous media. Modeling the effect of co-solvents on phase behavior is critical to CEOR reservoir simulations. The state-of-the-art is to use HLD (Hydrophilic Lipophilic Difference) with a modified form of NAC (Net Average Curvature) as an Equation of State (EoS) to model microemulsion phase behavior.

Aspects of the present disclosure relate to an improved method for predicting phase behavior in chemical enhanced oil recovery processes. Aspects of the present disclosure further relate to a method of performing a chemical enhanced oil recovery process based on the improved method for predicting phase behavior in chemical enhanced oil recovery processes. Aspects of the present disclosure relate to a method of designing a chemical solution for use in a chemical enhanced oil recovery process. Aspects of the present disclosure also relate to systems for implementing these methods.

The methods and systems disclosed herein advantageously predict phase behavior with a mean solubilization ratio being a direct function of hydrophilic-lipophilic difference (HLD). By way of contrast, in prior systems, the mean solubilization ratio is not a direct function of hydrophilic-lipophilic difference (HLD). Specifically, methods prior to the current approach (and indeed, prior to the HLD-NAC approaches above) considered net curvature (rather than net average curvature) as a function of HLD as shown in the following equation:

$${C}_{net} = \vert \frac{1}{{R}_{o}} \vert - \vert \frac{1}{{R}_{w}} \vert = - \frac{HLD}{L} \qquad ( 2 )$$

Such a limitation constrains the net curvature to be zero at optimum only, when HLD is zero. However, physically, net curvature of solubilized domains can be zero when there are equal amounts of oil and water solubilized in the microemulsion. Such a phenomenon is possible at all values of HLD (not just at zero).

The methods and systems also need not utilize the net curvature equation. Consequently, the methods and systems can advantageously and correctly capture the HLD physical phenomenon that net curvature of solubilized domains can be zero when there are equal amounts of oil and water solubilized in the microemulsion, which is possible at all values of HLD (not just at zero). Accordingly, in embodiments, the methods and systems conform with net curvature of solubilized domains being zero for at least one point other than at optimum salinity. Thus, the methods and systems reduce the error in phase behavior prediction associated with previous net curvature methods.

Aspects of the present disclosure also relate to methods and systems that accurately predict symmetrical two phase regions on ternary phase diagrams for oil, brine, and surfactant. In particular, the methods and systems result in a harmonic mean of solubilization at a specific HLD being constant, which results in symmetric two-phase regions. This defines clear phase boundaries without any discontinuity in phase behavior prediction, which makes computation in reservoir simulation more robust and efficient.

A method for predicting phase behavior of a microemulsion system in a chemical enhanced oil recovery process disclosed herein comprises determining a hydrophilic-lipophilic difference based on a ratio of salinity to optimum salinity in the microemulsion system. The method further comprises determining a mean solubilization ratio as a direct function of the hydrophilic-lipophilic difference at a same state as an optimum solubilization. The method also comprises predicting phase behavior based on the determined mean solubilization ratio.

A method of performing a chemical enhanced oil recovery process disclosed herein comprises predicting phase behavior of a microemulsion system in an oil reservoir comprising a surfactant, oil, and water from an experimental microemulsion system comprising the same surfactant formulation. The method further comprises injecting the surfactant formulation into the oil reservoir. For example, predicting phase behavior of a microemulsion system can involve determining a hydrophilic-lipophilic difference based on a ratio of salinity to optimum salinity in the experimental microemulsion system. This prediction step can further involve determining a mean solubilization ratio as a direct function of the hydrophilic-lipophilic difference at a same state as an optimum solubilization. Additionally, the prediction step can involve predicting phase behavior based on the determined mean solubilization ratio.

A system for performing a chemical enhanced oil recovery process disclosed herein incorporates a method for predicting phase behavior in chemical enhanced oil recovery processes. The system further comprises a processing unit configured to receive a data stream comprising experimental data from an experimental microemulsion system. Additionally, the system comprises a memory communicatively connected to the processing unit. The memory stores instructions which, when executed by the processing unit, cause the system to perform a method for predicting phase behavior in chemical enhanced oil recovery.

The optimum salinity S* and the optimum solubilization ratio σ* can be determined, for example, experimentally. Alternatively, the optimum salinity S* and the optimum solubilization ratio σ* can be determined by existing predictive correlations.

In certain embodiments of the methods and systems, predicting phase behavior facilitates planning subsequent experiment(s) for a single surfactant formulation. The data resulting from these subsequent experiment(s) can then be used to better predict phase behavior for the single surfactant formulation. Predicting phase behavior as disclosed herein as compared to existing methods (including HLD-NAC methods) can advantageously reduce the number of experiments necessary to accurately portray phase behavior for a combination of oil, water, and a single surfactant formulation. Reduction in the number of experiments reduces the cost to accurately predict phase behavior while also shortening the time to accurately predict phase behavior.

In other embodiments of the methods and systems, the surfactant formulation is injected into an oil reservoir. In these embodiments, predicting phase behavior facilitates increased oil production by better modeling of chemical enhanced oil recovery processes. Therefore, according to the methods and systems disclosed herein, it is possible to forecast field scale oil recovery for the surfactant.

Phase behavior prediction as disclosed herein arises from experiments conducted with a specific oil at a fixed state. Based on these experiments, the method can be used to predict phase behavior with other oils.

According to embodiments of the methods and systems disclosed herein, the mean solubilization ratio can be normalized to the optimum solubilization ratio. In embodiments, the hydrophilic-lipophilic difference is calculated as a natural logarithm of the ratio of salinity to optimum salinity.

Some embodiments of the methods and systems involve generating a symmetrical binodal curve on a ternary phase diagram of the microemulsion system representing a phase boundary between two-phase regions and a single phase region. The symmetrical binodal curve can represent the mean solubilization ratio at a specific salinity:

$${σ}_{avg} = \frac{2 {σ}_{o} {σ}_{w}}{{σ}_{o} + {σ}_{w}} \qquad ( 3 )$$

The steps of the methods disclosed herein can be executed on a computer system. For example, the step of determining a hydrophilic-lipophilic difference can be executed on a computing system. As another example, the step of determining the mean solubilization ratio can be executed on a computing system. As yet another example, the step of predicting phase behavior can be executed on a computing system.

The methods and systems disclosed herein can utilize the equation:

$$\frac{1}{{σ}_{avg}} ∝ \vert HLD \vert , \qquad ( 4 )$$

This equation implies that the mean solubilization ratio (σ_{avg}) normalized to the optimum solubilization ratio (σ*) is inversely proportional to the product of optimum solubilization ratio and HLD at the same state conditions. This equation is specific to each surfactant formulation.

The net curvature equation predicts phase behavior incorrectly. Alternatively, the harmonic average of solubilization ratio can be represented as a function of HLD, which forms the fundamental framework of an Equation of State. Strey, R. (1994). Microemulsion microstructure and interfacial curvature. Colloid and Polymer Science, 272(8), 1005-1019 (which is incorporated by reference in its entirety herein) showed that the average domain size is a function of temperature and is at its maximum at optimum conditions. Temperature is a state variable that is linearly related to HLD. From Strey's observations, like the average domain size, the average solubilization ratio is inversely proportional to the deviation from optimum (absolute value of HLD). Hence,

$$\frac{1}{{σ}_{avg}} ∝ \vert HLD \vert , \qquad ( 5 )$$

Equation (5), can be expressed as,

$$\frac{1}{{σ}_{avg}} = a \vert HLD \vert + b , \qquad ( 6 )$$

where a and b are constants.

At optimum (HLD=0), the average solubilization ratio is equal to the optimum solubilization ratio (σ*). Hence, the value of b is 1/σ*. Therefore Eq. (6) becomes

$$\frac{1}{{σ}_{avg}} = a \vert HLD \vert + \frac{1}{{σ}^{*}} . \qquad ( 7 )$$

When only the salinity (S) is changing in the system, with all other state variables fixed, HLD as a function of optimum salinity (S*) is expressed as

$$HLD {\vert}_{T , P , EACN , f ( A ) , Cc} = ln ( \frac{S}{{S}^{*}} ) . \qquad ( 8 )$$

Rearranging Eq. (7) and using Eq. (8) provides

$$\frac{{σ}_{avg}}{{σ}^{*}} = \frac{1}{a {σ}^{*} \vert ln ( \frac{S}{{S}^{*}} ) \vert + 1} . \qquad ( 9 )$$

Equation (9) implies that the average solubilization ratio normalized to the optimum solubilization ratio is inversely proportional to the product of optimum solubilization ratio and HLD. Equation (9) is a unique solution, specific to a surfactant formulation. Therefore, the unique solution tuned to experiments done with a specific oil at a fixed state can be used to predict phase behavior with other oils at different states of interest. This feature helps in designing and planning laboratory experiments.

The dimensionless HLD-NAC model presented by Ghosh, S. & and Johns, R. (2016). Dimensionless Equation of State to Predict Microemulsion Phase Behavior. Langmuir, 32(35), 8969-8979 (which is incorporated by reference in its entirety herein) produces symmetric solubilization curves when plotted in the HLD space. Chang et al. (2018). Structure-Property Model for Microemulsion Phase Behavior. SPE Improved Oil Recovery Conference. Society of Petroluem Engineers (which is incorporated by reference in its entirety herein) had demonstrated an attempt to fit the oil solubilization curve which compromised the fit of the brine solubilization curve and vice versa.

Equation (9) is subject to more flexibility with two different constants a⁺ and a⁻ instead of a single constant a. In ideal cases, a⁺ and a⁻ are equal and the equation is symmetrical around the optimum condition of HLD equal to zero. However, complex formulations used in enhanced oil recovery processes often do not exhibit symmetry due to the use of complex and mixed surfactants as well as incomplete equilibration. Asymmetry can be captured with different values of a⁺ and a⁻ to tune experimental data in over-optimum and under-optimum conditions, respectively.

Accordingly, some embodiments of the methods and systems involve generating an asymmetrical curve on a ternary phase diagram of the microemulsion system representing a phase boundary between two-phase regions and a single phase region. In these embodiments, the mean solubilization ratio can be determined as a direct function of the hydrophilic-lipophilic difference by the equations:

$$\begin{matrix}\frac{{σ}_{avg}}{{σ}^{*}} = \frac{1}{{a}^{+} {σ}^{*} \vert ln ( \frac{S}{{S}^{*}} ) \vert + 1} where S \geq {S}^{*} & ( 10 ) \\ \frac{{σ}_{avg}}{{σ}^{*}} = \frac{1}{{a}^{-} {σ}^{*} \vert ln ( \frac{S}{{S}^{*}} ) \vert + 1} where S < {S}^{*} & ( 11 )\end{matrix}$$

In these equations: S is salinity; S* is optimum salinity;

$$hydrophilic \text{-} lipophilic difference ( HLD ) = ln ( \frac{S}{{S}^{*}} ) ;$$

σ* is optimum solubilization ratio; σ_{avg} is the mean solubilization ratio; and a⁺ and a⁻ are fitting parameters that can aid in modeling asymmetrical phase behavior, if observed in experiments.

Optimum salinity and solubilization ratios are commonly determined experimentally. However, they can also be determined by already existing predictive correlations that determine the optimum conditions outside the range of experimental conditions using structure-performance relationships as demonstrated by Chang et al. (2018). Structure-Property Model for Microemulsion Phase Behavior. SPE Improved Oil Recovery Conference. Society of Petroluem Engineers (which is incorporated by reference in its entirety herein). Once optimum salinity and solubilization ratio are determined (from experiments or from correlations), Eqs. (10) and (11) can be used to determine σ_{avg} at a specific salinity. The average solubilization ratio σ_{avg} is

$${σ}_{avg} = \frac{2 {σ}_{1} {σ}_{2}}{{σ}_{1} + {σ}_{2}} = \frac{2 {C}_{13} {C}_{23}}{{C}_{33} ( {C}_{13} + {C}_{23} )} . \qquad ( 12 )$$

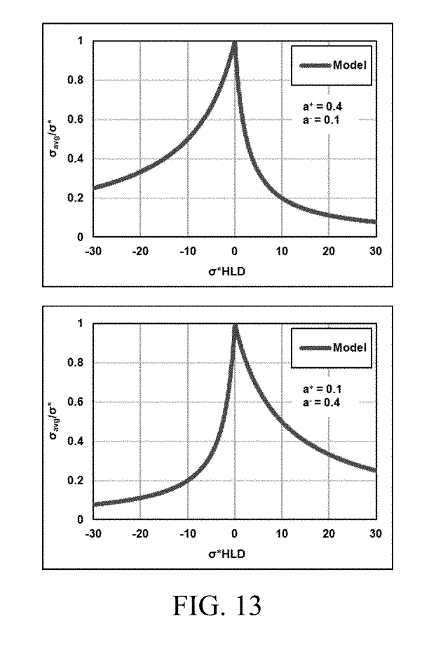

Hence, at a specific salinity, Eq. (12) is the locus of the two-phase region in the composition space. FIG. 13 shows example of asymmetrical model outputs from equations (10) and (11) using different values of a⁺ and a⁻.

The upper and lower HLD limits within which, a type III microemulsion can exist is represented as HLD_{U} and HLD_{L} respectively. The HLD equation can be used to get the specific state value of the limits. In prior Equation of State models (Acosta et al., 2003, Net-Average Curvature Model for Solubilization and Supersolubilization in Surfactant Microemulsions. Langmuir, 19(1), 186-195; Ghosh, S. & Johns, R., 2016, Dimensionless Equation of State to Predict Microemulsion Phase Behavoir. Langmuir, 32(35), 8969-8979; Khorsandi, S. & Johns, R., 2016, Robush Flash Calculation Algorithm for Microemulsion Phase Behavior. Journal of Surfactants and Detergents, 19(6), 1273-1287) (which are incorporated by reference in their entireties herein), these limits could be calculated as a model output using the net curvature equation. HLD_{U} and HLD_{L} are treated as model inputs. When a system with more than one surfactant is considered, mixing rules for HLD as described in Magzymov et al. (2016). Impact of Surfactant Mixtures on Microemulsion Phase Behavior. SPE Annual Technical Conference and Exhibition. Society of Petroleum Engineers; Acosta, E. & Bhakta, A. (2009). The HLD-NAC Model for Mixtures of Ionic and Nonionic Surfactants. Journal of Surfactants and Detergents, 12(1), 7-19; and Ghosh, S. & Johns, R. (2018). A Modified HLD-NAC Equation of State to Predict Alkali/Surfactant/Oil/Brine Phase Behavior. SPE Journal, 23(02), 550-566 (which are incorporated by reference in their entireties herein) may be extended to calculate HLD_{U} and HLD_{L} for mixtures. Considered below is one surfactant component.

The excess phases in the two and three phase systems are assumed pure. Hence, once the phase behavior boundary is obtained, the following checks and calculations can be done to predict phase behavior at a given overall composition:

1. Based on the overall composition, calculate the average solubilization ratio assuming single phase microemulsion σ_{avg}¹.
2. Calculate σ_{avg} for a specific HLD from Eqs. (9) and (10).
3. If σ_{avg}¹<σ_{avg}, conclude that a single phase microemulsion is formed with composition which is the same as the overall composition.
4. If σ_{avg}¹>σ_{avg}, conclude that more than one phase exists and determine the type, phase volumes and phase composition (phase behavior) following the procedure:
a. If HLD<HLD_{L}, a type I microemulsion exists with a pure excess oil phase and σ₁ is known. Equation (11) is then used to calculate σ₂.
b. If HLD>HLD_{U}, a type II microemulsion exists with a pure excess brine phase and σ₂ is known. Equation (11) is then used to calculate σ₁.
c. The type I and type II compositions represented by C₁₃, C₂₃ and C₃₃ are obtained by solving the simple system of linear equations (13), (14) and (15) with the solubilization ratios from steps 4a and 4b.

C₁₃−σ₁C₃₃=0 (13)

C₂₃−σ₂C₃₃=0, (14)

C₁₃+C₂₃+C₃₃=1, (15)

d. If HLD_{L}≤HLD≤HLD_{U}, existence of three microemulsion types are possible; type I, type II or type III. We first determine the type III microemulsion composition at the invariant point as follows:
i. Condition 1: C₃₃ in a type III microemulsion at optimum is

$${C}_{33}^{opt} = \frac{1}{2 {σ}^{*} + 1} . \qquad ( 16 )$$

ii. Condition 2: C₃₃ in a type III microemulsion at the limits HLD_{L} and HLD_{U} is zero.
iii. C₃₃ in type III for HLD_{L}≤HLD≤HLD_{U} is obtained from linear interpolations between Condition 1 and Condition 2.
iv. Solving the average solubilization equation (12) and conserving mass using equation (15) provides a quadratic equation with roots as shown in equation (17)
   C₁₃ or

$${C}_{23} = \frac{- ( {C}_{33} - 1 ) \pm \sqrt{{( {C}_{33} - 1 )}^{2} - 2 {σ}_{avg} ( {C}_{33} - {C}_{33}^{2} )}}{2} \qquad ( 17 )$$

v. If the system is under optimum (HLD<0) the larger quadratic root from equation (17) is C₁₃ and the smaller root is C₂₃.
vi. If the system is over optimum (HLD>0) the larger quadratic root from equation (17) is C₂₃ and the smaller root is C₁₃.
vii. If the system is at optimum, (HLD=0), the type III microemulsion (invariant point) has equal solubilization of oil and brine. Hence,

$${C}_{13} = {C}_{23} = \frac{1 - {C}_{33}}{2} = \frac{2 {σ}^{*}}{2 {σ}^{*} + 1} \qquad ( 18 )$$

viii. After the type III invariant point is determined, the tie triangle can be constructed and the type I and II regions can be determined.
   e. With the composition of microemulsion and the phase behavior boundaries known, phase quantities can be computed by conserving the mass of components in the system.
   In summary, once the optimum salinity and solubilization ratio for a formulation at various states are known, the model parameters a⁺, a⁻, HLD_{U} and HLD_{L} are sufficient to define the Equation of State.

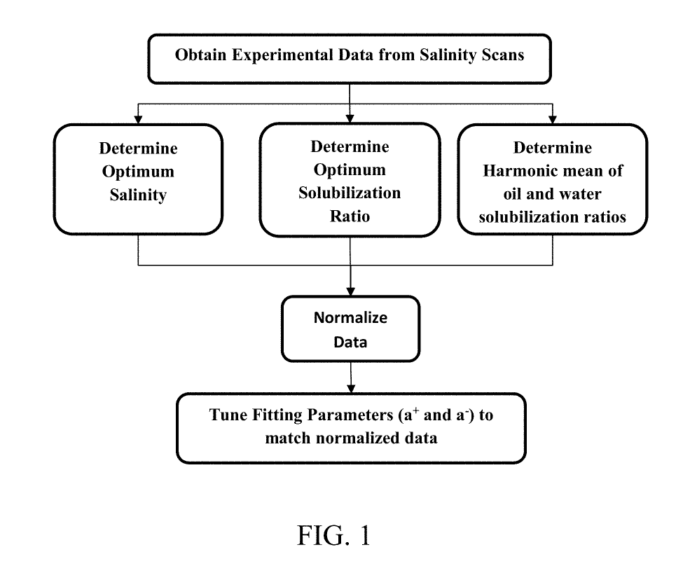

According to aspects of the present disclosure, FIG. 1 provides a flow chart illustrating steps to determine fitting parameters for surfactant solutions based on experimental data. First salinity scans are taken for surfactant formulations being screened for use in chemical enhanced oil recovery processes. Healy et al. (Multiphase microemulsion systems. Society of Petroleum Engineers Journal, 1976, 16(03), pp. 147-160) (which is incorporated by reference in its entirety herein), outlines a screening procedure for conducting salinity scans for surfactant formulations. The optimum salinity and optimum solubilization ratio can then be determined as outlined by Ghosh et al., An Equation-of-State Model to Predict Surfactant/Oil/Brine-Phase Behavior. SPE Journal. 21(04), pp. 106-125. (2016) (which is incorporated by reference in its entirety herein). The harmonic mean of oil and water solubilization ratios can also be determined as described herein. Normalized data from the salinity scans can be plotted and the fitting parameters (a⁺ and a⁻) of the above equations can be tuned to match the normalized data.

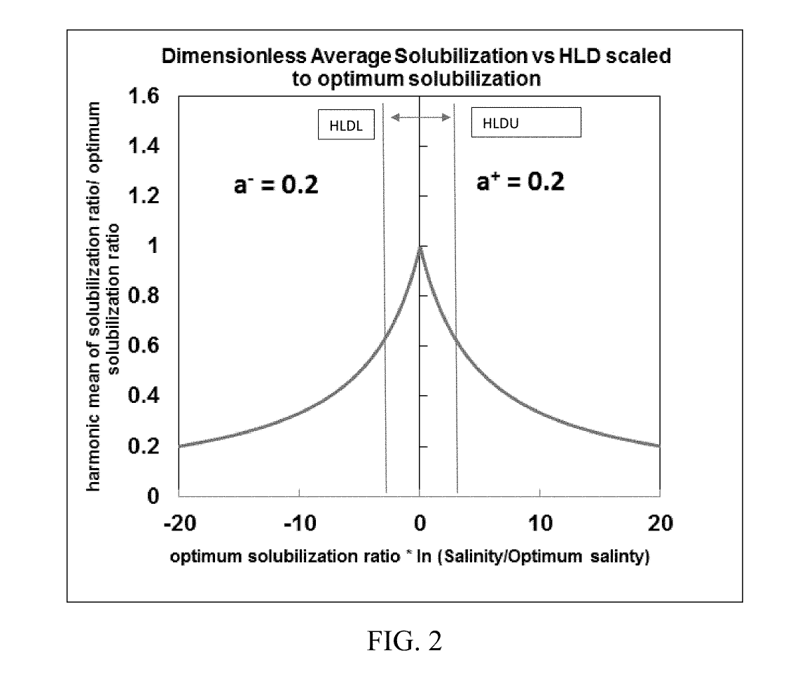

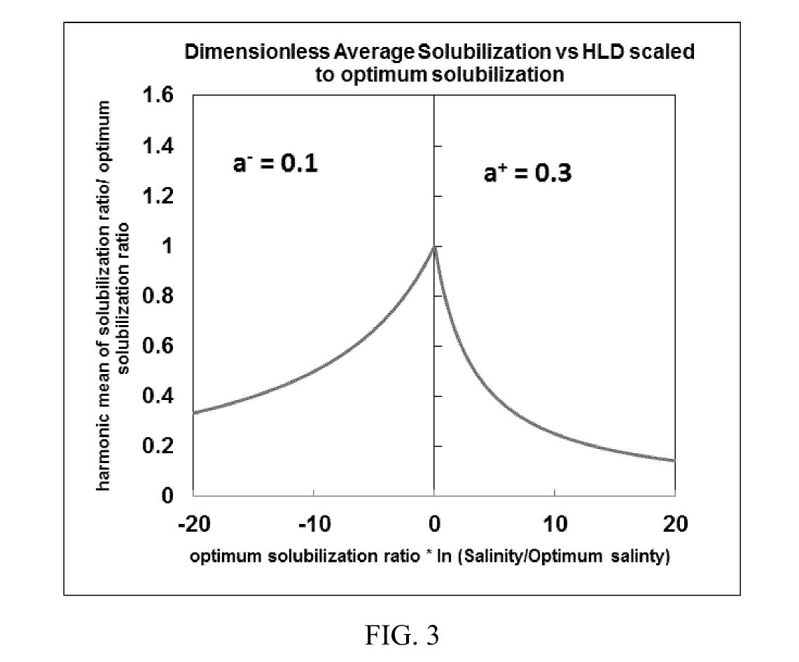

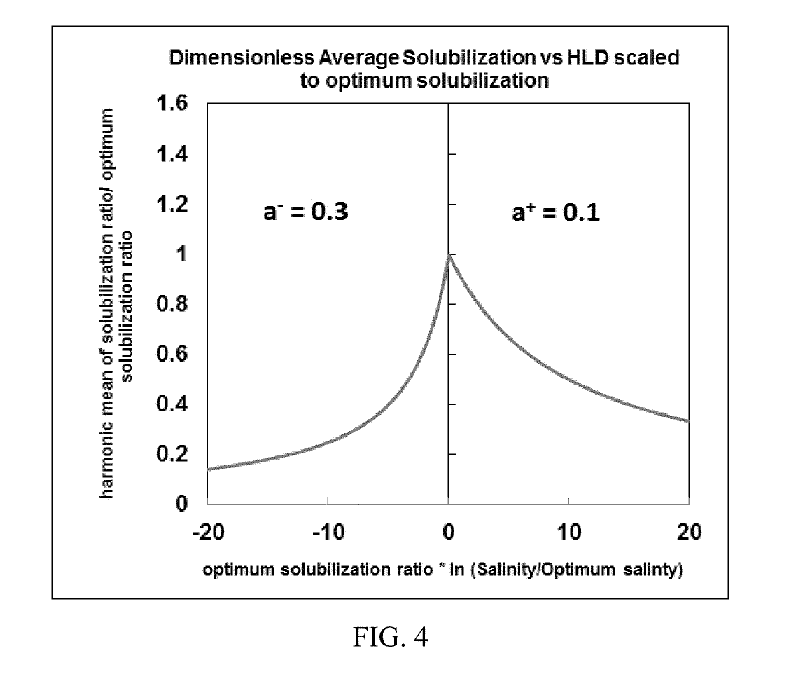

FIGS. 2-4 show exemplary symmetrical and asymmetrical functions of dimensionless average solubilization versus hydrophilic-lipophilic difference (HLD) scaled to optimum solubilization using different values of fitting parameters. In particular, FIG. 2 shows a symmetrical case modeled by having fitting parameters a⁻ and a⁺ at a constant of 0.2. FIG. 3 shows an asymmetrical case modeled by having fitting parameter a equal to 0.1 and fitting parameter a⁺ equal to 0.3. FIG. 4 shows an asymmetrical case modeled by having fitting parameter a⁻ equal to 0.3 and fitting parameter a⁺ equal to 0.1.

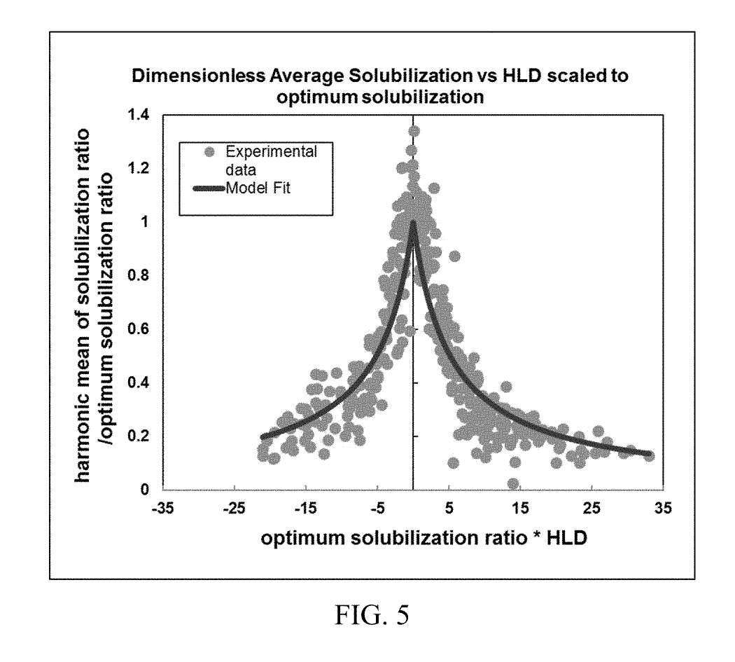

As shown in FIG. 5, the methods and systems disclosed herein can advantageously describe multiple phase behavior experiments (i.e., salinity scans) using one mathematical model. For example, FIG. 5 illustrates a model of 80 experiments with 872 data points where the harmonic mean of the solubilization ratio/the optimum solubilization ratio as a function of the product of the optimum solubilization ratio and hydrophilic-lipophilic difference (HLD) accurately models that experimental data. In FIG. 5, the model is a symmetrical case where fitting parameters, a⁺ and a⁻, are equivalent.

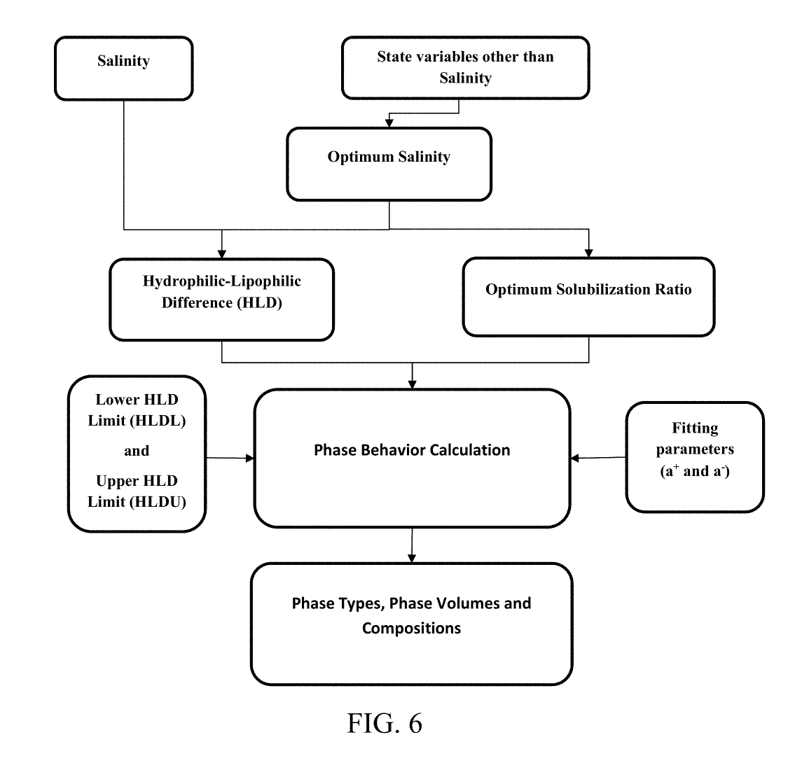

Once the parameters a⁺ and a⁻ are determined, the phase behavior calculation protocol can be used to determine the phase types, volumes and composition as a function of state variables in a reservoir simulator. The methods and systems disclosed herein can further determine a number of phases and phase volumes in the microemulsion system. The methods and systems can further determine composition(s) of the phase(s). FIG. 6 shows the inputs and outputs used by a reservoir simulator for predicting phase behavior.

In some embodiments, other chemicals (e.g., co-solvents, alkali) are used with surfactants in chemical enhanced oil recovery processes. A separate partitioning model can be coupled with the above described methods/systems to capture the effect of the other chemicals on the phase behavior in equation of state (EOS) flash calculations. For example, a Prouvost-Pope-Rouse model can be used to capture co-solvent partitioning across oil, brine and microemulsion phases. The coupling method described in Ghosh and Johns, A Modified HLD-NAC Equation of State to Predict Alkali-Surfactant-Oil-Brine Phase Behavior, SPE Annual Technical Conference and Exhibition, 2015 (which is incorporated by reference in its entirety herein) can be utilized for coupling alkali with the disclosed methods and systems.

In order to determine the number of phases in the microemulsion system, a mean solubilization ratio σ¹_{avg} can be calculated assuming a single phase microemulsion system and a mean solubilization ratio σ_{avg} can be calculated. Then it can be determined whether σ¹_{avg} is less than σ_{avg} or σ¹_{avg} is greater than σ_{avg}. σ¹_{avg} being less than σ_{avg} provides the conclusion that the microemulsion system is a single phase system. σ¹_{avg} being greater than σ_{avg} provides the conclusion that the microemulsion system is a multiphase system.

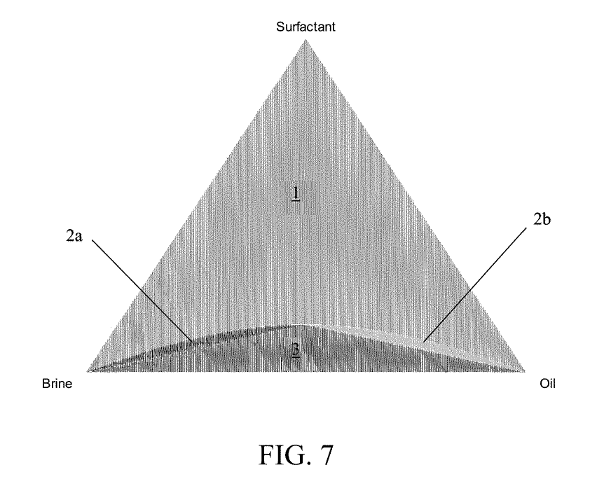

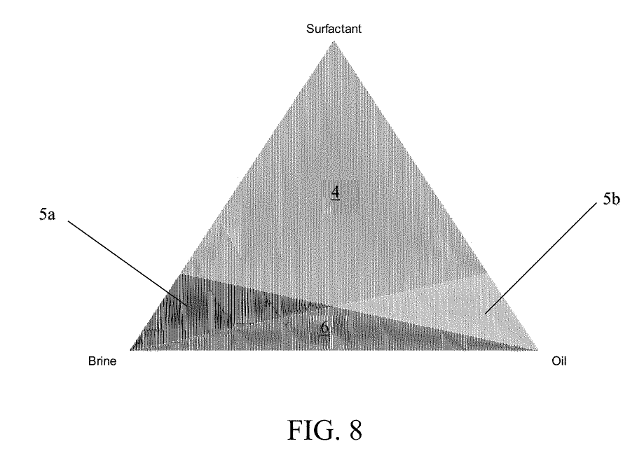

An exemplary symmetrical binodal curve is illustrated in FIG. 7. In FIG. 7, there is a single phase region 1, two two-phase regions 2a, 2b, and a three-phase region 3. The two-phase regions 2a, 2b are curved and symmetrical. These two-phase regions 2a, 2b are physical and robust for simulations. By contrast, FIG. 8 provides an exemplary representation of a prior HLD-NAC model. In FIG. 8, there is a single phase region 4, two two-phase regions 5a, 5b, and a three-phase region 6. The two-phase regions 5a, 5b are triangular. These two-phase regions not physical and are not robust for simulation.

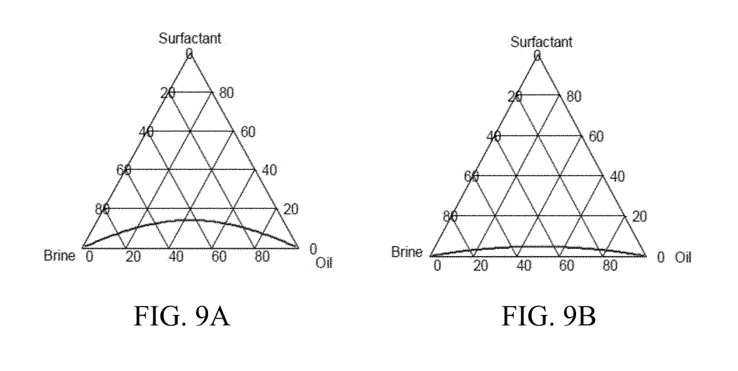

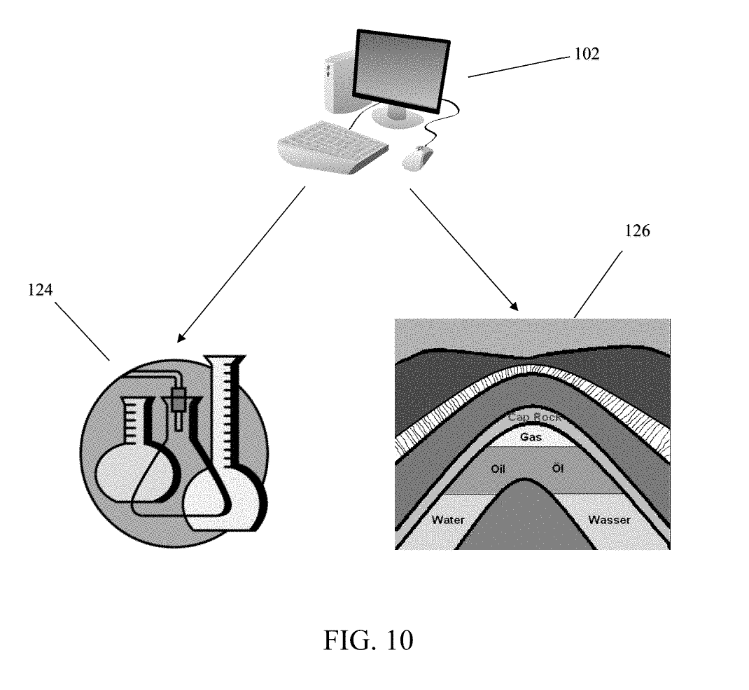

The mean solubilization ratio at a specific salinity forms the locus of the two-phase region in a composition space. For example, FIG. 9A shows a symmetric binodal curve for a mean solubilization ratio of 3 cc/cc and FIG. 9B shows a symmetric binodal curve for a mean solubilization ratio of 10 cc/cc.

Determining the number of phases in the microemulsion system can further comprise providing an upper HLD limit (HLDU); providing a lower HLD limit (HLDL): and determining whether a two-phase system exists or a three-phase system exists based on comparing HLD to the lower HLD limit and/or the upper HLD limit.

For example, HLD being less than the lower HLD limit provides the conclusion that a two-phase system exists having a microemulsion phase and an excess oil phase. If HLD is less than the lower HLD limit, the water solubilization ratio can be calculated from the overall composition; the oil solubilization ratio can be calculated from the equation:

$${σ}_{avg} = \frac{2 {σ}_{o} {σ}_{w}}{{σ}_{o} + {σ}_{w}} ;$$

and phase volumes can be calculated based on the water solubilization ratio and the oil solubilization ratio.

As another example, HLD being greater than the lower HLD limit provides the conclusion that a two-phase system exists having a microemulsion phase and an excess brine phase. If HLD is greater than the lower HLD limit, the oil solubilization ratio can be calculated from the overall composition; the water solubilization ratio can be calculated from the equation:

$${σ}_{avg} = \frac{2 {σ}_{o} {σ}_{w}}{{σ}_{o} + {σ}_{w}} ;$$

and phase volumes can be calculated based on the water solubilization ratio and the oil solubilization ratio.

As yet another example, the lower HLD limit ≤HLD≤the upper HLD limit provides the conclusion that a three-phase system exists having a microemulsion phase, an excess oil phase, and an excess brine phase. If the lower HLD limit ≤HLD≤the upper HLD limit, a composition of the three-phase system can be determined at an invariant point by determining an optimum concentration of surfactant with the equation:

$$\frac{1}{( 2 σ * + 1 )}$$

linearly interpolating a concentration of surfactant between the optimum concentration and zero; determining a tie triangle; and separating a first two-phase system having the microemulsion phase and the excess oil phase and a second two-phase system having the microemulsion phase and the excess brine phase.

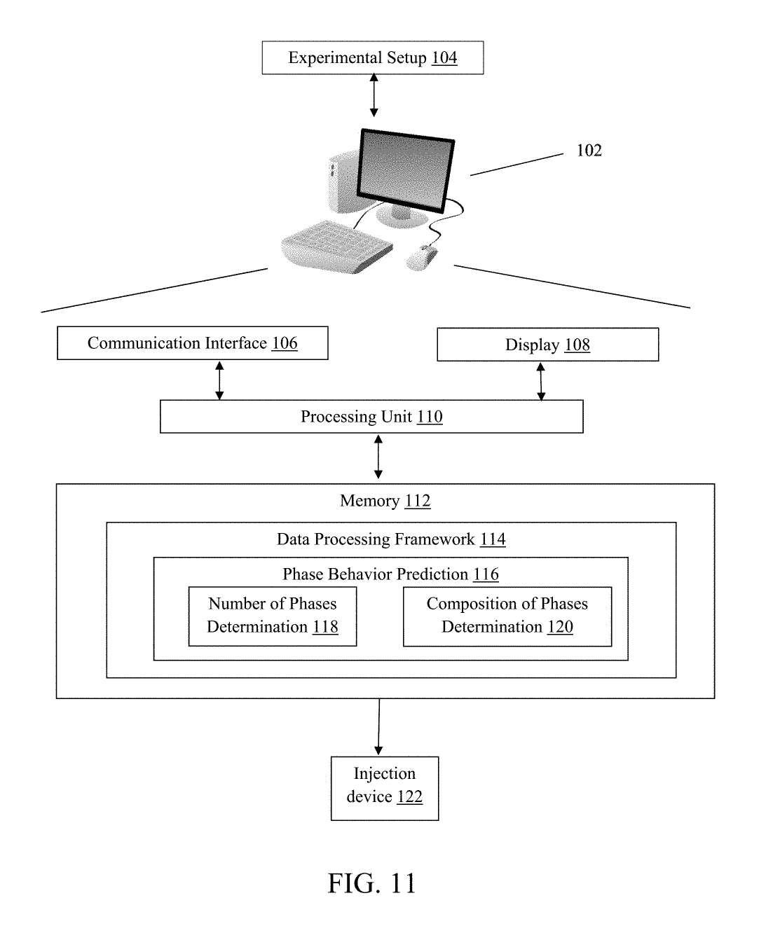

FIG. 11 depicts an exemplary embodiment of a system for performing a chemical enhanced oil recovery process.

In the embodiment shown, the computing system 102 includes a processing unit 110 and a memory 112. The processing unit 110 can be any of a variety of types of programmable circuits capable of executing computer-readable instructions to perform various tasks, such as mathematical and communication tasks.

The memory 112 can include any of a variety of memory devices, such as using various types of computer-readable or computer storage media. A computer storage medium or computer-readable medium may be any medium that can contain or store the program for use by or in connection with the instruction execution system, apparatus, or device. In example embodiments, the computer storage medium is embodied as a computer storage device, such as a memory or mass storage device. In particular embodiments, the computer-readable media and computer storage media of the present disclosure comprise at least some tangible devices, and in specific embodiments such computer-readable media and computer storage media include exclusively non-transitory media.

The computing system 102 can also include a communication interface 106 configured to receive data streams from an experimental setup 104. The experimental setup 104 is configured to perform phase behavior experiments on an experimental microemulsion system and generate phase behavior data. The computing system 102 is also configured to transmit notifications as generated by the data processing framework 114 and also includes a display 108 for presenting a user interface associated with the data processing framework 114. In various embodiments, the computing system 102 can include additional components, such as peripheral I/O devices, for example to allow a user to interact with the user interfaces generated by the data processing framework 114.

The data processing framework 114 of the exemplary embodiment includes a phase behavior prediction module 116 that executes a method of predicting phase behavior. As depicted, the phase behavior prediction module 116 includes two modules: (1) a module 118 for determining a number of phases in the microemulsion system; and (2) a module 120 for determining composition(s) of phase(s) in the microemulsion system. The exemplary system further includes an injection device 122.

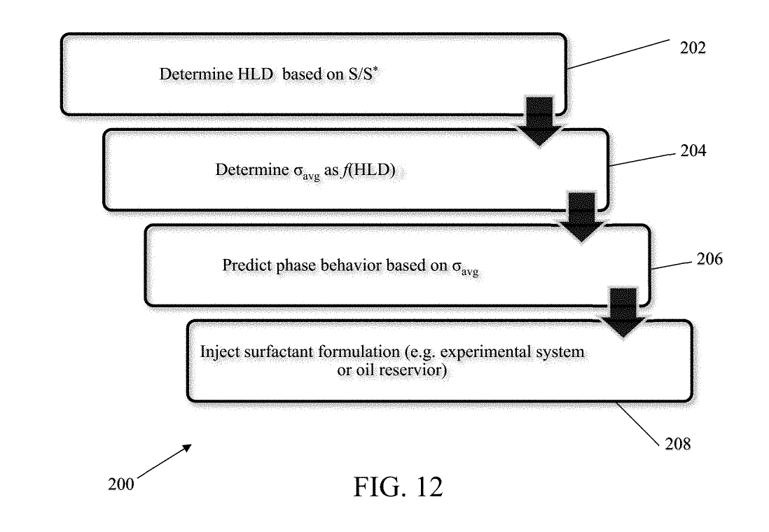

Turning to FIG. 12, FIG. 12 pictorially illustrates steps of a method 200 for predicting phase behavior disclosed herein in the form of a flow chart. As shown in the flow chart, step 202 involves determining a hydrophilic-lipophilic difference based on a ratio of salinity to optimum salinity in the microemulsion system. Step 204 involves determining a mean solubilization ratio as a direct function of the hydrophilic-lipophilic difference at a same state as an optimum solubilization. Step 206 involves predicting phase behavior based on the determined mean solubilization ratio. Step 208 involves performing additional experiments with the surfactant formulation or injecting it into an oil reservoir.

Embodiments of the present disclosure can be implemented as a computer process (method), a computing system, or as an article of manufacture, such as a computer program product or computer readable media. The computer program product may be a computer storage media readable by a computer system and encoding a computer program of instructions for executing a computer process. Accordingly, embodiments of the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system.

Exemplary advantages of the methods and systems disclosed herein include:
1. An inverse relationship between the normalized average solubilization ratio (σ_{avg}/σ*) and the product of the HLD and σ* exists for a surfactant formulation. Such a relationship, along with the HLD equation and correlations for σ*, can be used to predict phase behavior across a wide range of conditions.
2. The average solubilization ratio adequately defines the boundary between the single and two-phase regions in a pseudo ternary composition space.
3. The evolution of the three-phase region with changing state variables can be adequately represented by defining constant upper and lower HLD limits for a type III microemulsion as model inputs.
4. The HLD-AST model can be coupled with a co-solvent partitioning model to adequately represent experimental data.
5. The model is robust across the composition space which is critical to achieve convergence in mechanistic reservoir simulations.

### EXAMPLES

The following shows experimental validation and the modeling of pseudo ternary phase behavior.

Equation of State Prediction with Changing EACNs and Temperatures

The method of conducting salinity scan experiments to obtain microemulsion phase behavior data using glass pipettes is described elsewhere (Levitt et al., 2009, Identification and Evaluation of High-Performance EOR Surfactants. SPE Reservoir Evaluation & Engineering, 12(02), 243-253; Flaaten et al., 2009, A Systematic Laboratory Approach to Low-Cost, High-Performance Chemical Flooding. SPE Reservoir Evaluation & Engineering, 12(05), 713-723 (which are incorporated by reference in their entireties herein)). To summarize, aqueous surfactant solution, brine and oil are combined and mixed in desired proportions in graduated 5 ml borosilicate glass pipettes that are then sealed. The least count of the pipettes is 0.1 ml. The pipettes are then maintained at the desired temperatures in an oven and allowed to equilibrate over time. The interfaces formed because of phase separation are measured, which allows for an inference of the solubilization ratios (assuming excess phases are pure and the density of surfactant is the same as the density of brine).

In experiments, the surfactant formulation consisted of 1.5% (v/v) of an alkyl aryl sulfonate, 0.5% (v/v) of an isomerized olefin sulfonate resulting in total of 2% (v/v) aqueous surfactant concentration. Ethyl glycol mono-butyl ether was used as a co-solvent at 2.8% (v/v) of aqueous concentration. The initial conditions in all pipettes were the same with oil being 50% (v/v) of the total fluid mixture. Five different alkanes were used namely, dodecane (EACN=12), tetradecane (EACN=14), pentadecane (EACN=15), hexadecane (EACN=16) and octadecane (EACN=18). Measurements were done at four different temperatures, 65° C., 75° C., 85° C. and 95° C. Additionally, the 20 phase behavior experiments were repeated four times, resulting in a total of 80 experiments with 872 pipette measurements.

The logarithm of optimum salinity ln(S*), and the inverse of optimum solubilization ratios (σ*) were correlated using the linear relationship presented in Ghosh, S. & Johns, R. (2016). An Equation-of-State Model to Predict Surfactant/Oil/Brine-Phase Behavoir. SPE Journal, 21(04), 1106-1125 (which is incorporated by reference in its entirety herein). The correlation for the described experimental dataset is

$$\frac{1}{{σ}^{*}} = 0.061 ln ( {S}^{*} ) + 0.208 . \qquad ( 19 )$$

Furthermore, the HLD theory was used to find a correlation of ln(S*) as a function of the state variables changed in the experiments (EACN and T) as follows,

ln(S*)=0.15 EACN+0.0034(T−25)−4.62. (20)

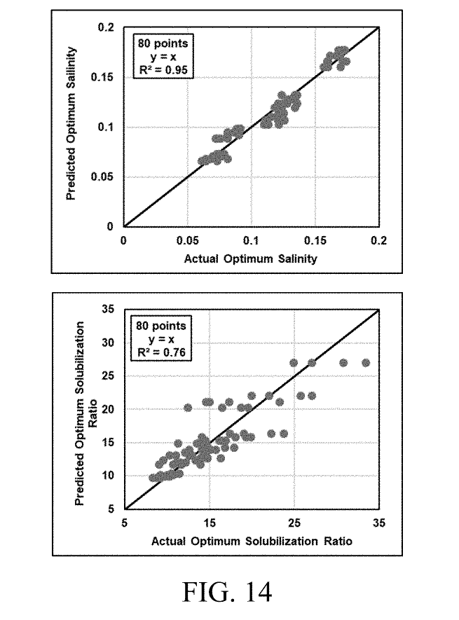

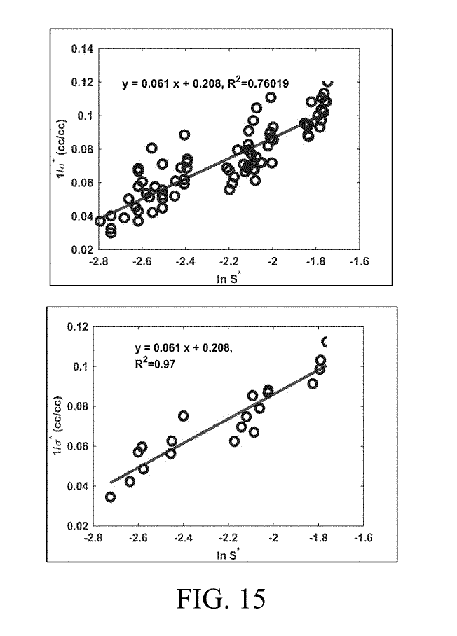

FIGS. 14 and 15 show the validity of correlations described in Eqs. (19) and (20). The optimum salinity correlation Eqs. (20) has a high coefficient of determination (R²=0.95). In comparison, the coefficient of determination for the predicted optimum solubilization ratios (σ*) is low. However, averaging the experimental data from the repetitions helped improving the R² to 0.97. While the determination of optimum salinities is relatively precise, the measurement technique of solubilization ratio is subject to large uncertainties.

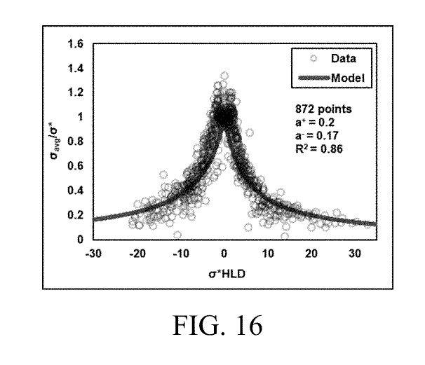

The solubilization ratios inferred from the experiments were used to calculate the average solubilization ratios using Eq. (12). The normalized average solubilization (σ_{avg}/σ*) for the 872 data points were matched to model equations (10) and (11) to determine the model parameters a⁺ and a⁻ as shown in FIG. 16.

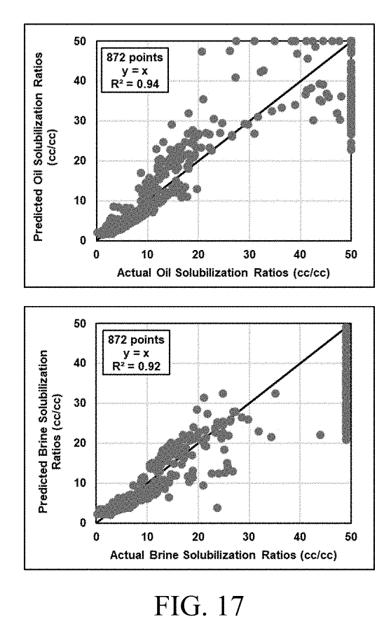

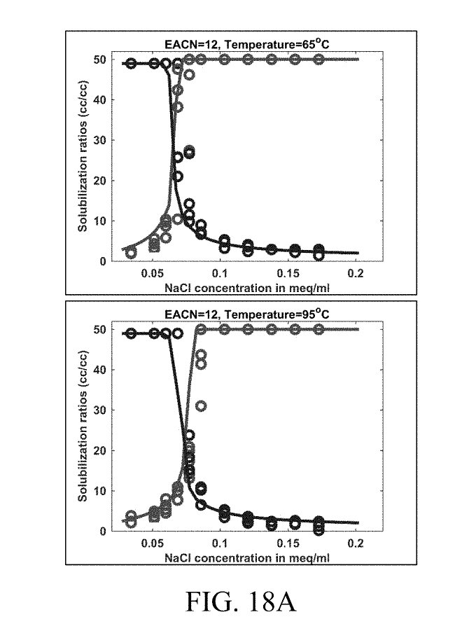

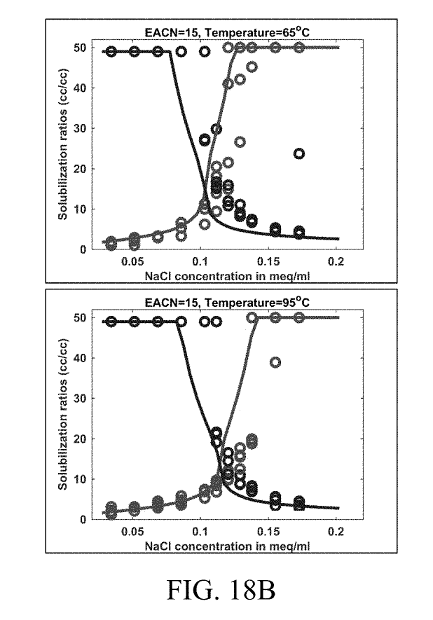

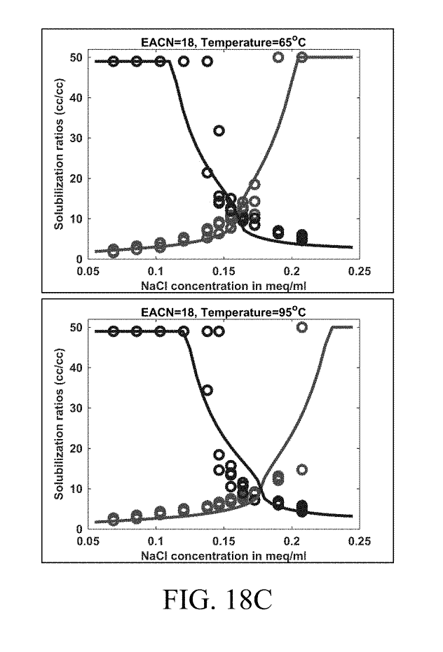

The limits HLD_{L} and HLD_{U} were the remaining model parameters that were determined by fitting the model predictions of oil and brine solubilization ratios to the experimental data. The resulting upper and lower HLD limits were 0.4 and −0.6 respectively. FIG. 17 shows a strong agreement between the model prediction and the actual experimental data. The coefficient of determination was above 0.9 for both oil and brine solubilization ratios. FIGS. 18A-C show examples of model predictions for six cases (out of the total 20 experiments).

HLD-AST Coupled with Co-Solvent Model

Hirasaki, G. (1982). Interpretation of the Change in Optimal Salinity with Overall Surfactant Concentration. Society of Petroleum Engineers Journal, 22(06), 971-982 (which is incorporated by reference in its entirety herein) modeled co-solvent partitioning between the microemulsions and the excess phases using constant partitioning coefficients. However, it was later determined that the partitioning of co-solvent is a function of the overall composition (Prouvost et al., 1985, Microemulsion Phase Behavoir: A Thermodynamic Modeling of the Phase Partitioning of Amphiphilic Species. Society of Petroleum Engineers Journal, 25(05), 693-703 (which is incorporated by reference in its entirety herein)). Prouvost's pseudophase model which calculates alcohol partitioning coefficients for up to two co-solvents, described in detail in Delshad, M., Pope, G. A., & Sepehrnoori, K. (2000). UTCHEM version 9.0 technical documentation, Center for Petroleum and Geosystems Engineering, The University of Texas at Austin, Austin, Tex., 78751 (which is incorporated by reference in its entirety herein) is used. The pseudophase model calculates

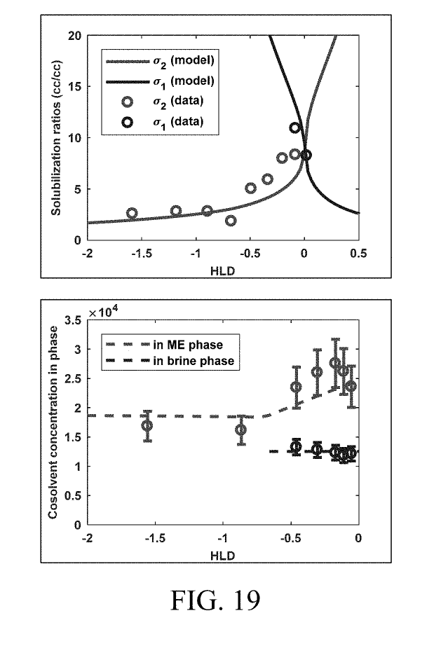

CPᵢ=Cᵢ+C_{a1,i}+C_{a2,i}, (21)

where C_{a1,i} and C_{a2,i} are the concentrations of co-solvent a₁ and a₂ associated to component i. The overall composition represented by the pseudo-associated components (CPᵢ) is then used as an input in the phase behavior model. Dwarakanath et al. (2008). Using Co-solvents to Provide Gradients and Improve Oil Recovery During Chemical Flooding in a Light Oil Reservoir. SPE Symposium on Improved Oil Recovery. Society of Petroleum Engineers (which is incorporated by reference in its entirety herein) measured the co-solvent partitioning, specifically the concentration of co-solvent in the excess brine and microemulsion phase, as a function of the phase behavior as seen in a salinity scan, which is used to validate the approach. Dwarakanath et al. (2008) Using Co-solvents to Provide Gradients and Improve Oil Recovery During Chemical Flooding in a Light Oil Reservoir. SPE Symposium on Improved Oil Recovery. Society of Petroleum Engineers (which is incorporated by reference in its entirety herein) used a single co-solvent system. FIG. 19 shows the phase behavior match using the model parameters summarized in Table 1.

TABLE 1
Summary of model parameters used in FIG. 19

| Variable | Description | Value |
|---|---|---|
| a⁺ | HLD-AST model parameter | 0.2 |
| a⁻ | HLD-AST model parameter | 0.1 |
| HLD_{L} | Lower HLD limit | −1.3 |
| HLD_{U} | Upper HLD limit | 1.3 |
| σ* | Optimum solubilization ratio | 9 |
| K_{w1} | Partition coefficient of monomeric co-solvent 1 | 2 |
| | between pseudophases 1 and 2 | |
| K_{M1} | Partition coefficient of monomeric co-solvent 1 | 60 |
| | between pseudophases 3 and 2 | |
| K₁ | Self-association constant of monomeric co-solvent 1 in | 35 |
| | pseudophase 2 | |
| A₁ | Ratio of molar volume of monomeric alcohol 1 to | 0.3 |
| | equivalent molar volume of surfactant | |

### Robust Phase Behavior Modeling

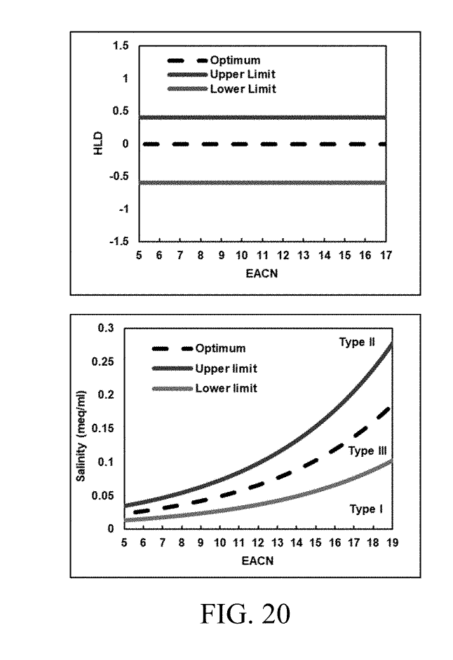

The new model using the average solubilization theory specifies HLD_{U} and HLD_{L}, that represent the upper and lower HLD limits, as constant model inputs. The evolution of the upper and lower salinity limits consequently is shown in FIG. 20. The model prediction agrees with the experimental trends observed by Salager et al. (1979), Optimum Formulation of Surfactant/Water/Oil Systems for Minimum Interfacial Tension of Phase Behavior. Society of Petroleum Engineers Journal, 19(02), 107-115 (which is incorporated by reference in its entirety herein).

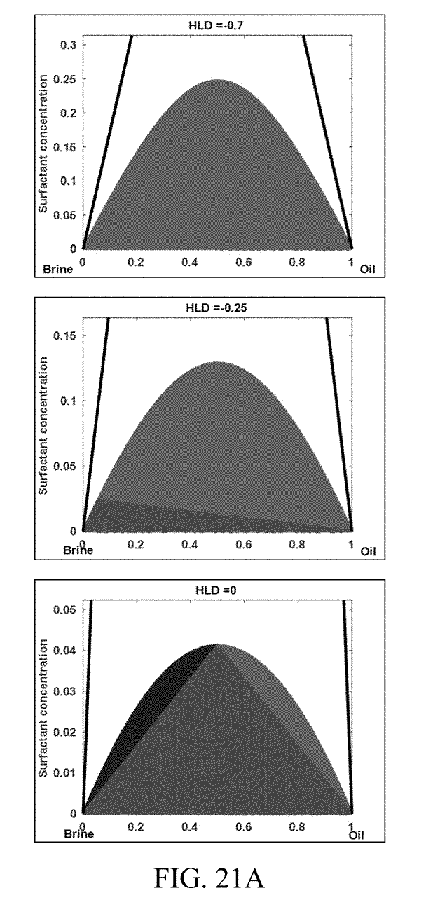

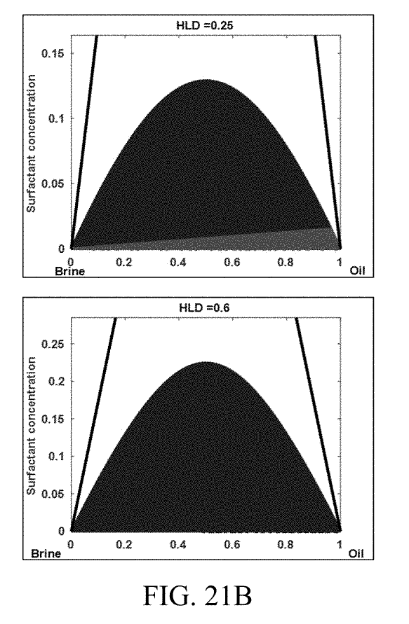

To demonstrate phase behavior modeling using HLD-AST in the ternary space, the tuned model parameters were used as determined by tuning 80 experiments (summarized in Table 2) and an optimum solubilization ratio of 10 cc/cc. FIGS. 21A-B show the evolution of clear phase behavior regions covering all four Winsor types of microemulsions typically seen in a salinity scan, as a function of HLD. A single type I region exists when the system is under optimum and HLD is less than HLD_{L}. As the HLD of the system increases, and the three-phase region (between the upper and lower limits) is entered, the evolution of the type III tie triangle can be clearly seen, existing with the type I and II lobes. Eventually, as the system moves beyond HLD_{U} to over optimum conditions, only a type II region exists. Clear definition of phase boundaries and flash criteria makes the model robust across the entire composition space.

TABLE 2
Summary of model parameters used in FIGS. 21A-B

| Variable | Description | Value |
|---|---|---|
| a⁺ | HLD-AST model parameter | 0.2 |
| a⁻ | HLD-AST model parameter | 0.17 |
| HLD_{L} | Lower HLD limit | −0.6 |
| HLD_{U} | Upper HLD limit | 0.4 |
| σ* | Optimum solubilization ratio | 10 |

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media. Further, the disclosed methods' stages may be modified in any manner, including by reordering stages and/or inserting or deleting stages, without departing from the overall concept of the present disclosure.

The present disclosure provides a complete description of the methods and systems. Since many embodiments of the methods and systems can be made without departing from the spirit and scope of the invention, the invention resides in the claims hereinafter appended.

### Abbreviations

a, a⁻, a⁺=fitting parameters to match mean solubilization ratio as a function of HLD
HLD=hydrophilic-lipophilic difference
S=salinity
S*=optimum salinity
σ*=optimum solubilization ratio
σ_{avg}=mean solubilization ratio
σₒ=oil solubilization ratio
σ_{w}=water solubilization ratio
C_{c}=Characteristic curvature of surfactant
Cᵢⱼ=Concentration of component i in phase j
EACN=Equivalent Alkane Carbon Number (dimensionless)
fA=function of co-solvent type and concentration
HLD=Hydrophilic lipophilic difference
K=Slope of dependence of HLD on EACN (dimensionless)
L=Surfactant length parameter (Å)
P=Pressure (bars)
Rᵢ=Radius of curvature of micelle (i=1 for brine, 2 for oil) (Å)

### T=Temperature (° C.)

α=Slope of dependence of HLD on T (° C.⁻¹)
β=Slope of dependence of HLD on P (bar⁻¹)
σᵢ=Solubilization ratio (i=1 for brine, 2 for oil) (cc/cc)
ξ=Average hydrodynamic radius of solubilized system (Å)
avg=average
L=Lower limit
U=Upper limit
*=optimum state

1. A method for predicting phase behavior of a microemulsion system in a chemical enhanced oil recovery process, the method comprising:
determining a hydrophilic-lipophilic difference based on a ratio of salinity to optimum salinity in the microemulsion system;
determining a mean solubilization ratio as a direct function of the hydrophilic-lipophilic difference; and
predicting phase behavior based on the determined mean solubilization ratio.

2. The method of claim 1, wherein the mean solubilization ratio is normalized to the optimum solubilization ratio.

3. The method of claim 1, wherein the hydrophilic-lipophilic difference is calculated as a natural logarithm of the ratio of salinity to optimum salinity.

4. The method of claim 1, wherein the method allows net curvature of solubilized domains to be zero at all salinity values.

5. The method of claim 1, further comprising generating a symmetrical binodal curve on a ternary phase diagram of the microemulsion system representing a phase boundary between two-phase regions and a single phase region.

6. The method of claim 1, wherein only symmetrical binodal curves on ternary phase diagrams of the microemulsion system representing a phase boundary between two-phase regions and a single phase region are generated.

7. The method of claim 1, wherein the step of determining a hydrophilic-lipophilic difference is executed on a computing system.

8. The method of claim 1, wherein the step of determining the mean solubilization ratio is executed on a computing system.

9. The method of claim 1, wherein the step of predicting phase behavior is executed on a computing system.

10. The method of claim 1, wherein the mean solubilization ratio is determined as a direct function of the hydrophilic-lipophilic difference by the equations:
$$\frac{{σ}_{avg}}{{σ}^{*}} = \frac{1}{{a}^{+} {σ}^{*} \vert ln ( \frac{S}{{S}^{*}} ) \vert + 1} when S \geq {S}^{*}$$
$$\frac{{σ}_{avg}}{{σ}^{*}} = \frac{1}{{a}^{-} {σ}^{*} \vert ln ( \frac{S}{{S}^{*}} ) \vert + 1} when S < {S}^{*}$$
where
S is salinity;
S* is optimum salinity;
$$hydrophilic - lipophilic difference ( HLD ) = ln ( \frac{S}{{S}^{*}} )$$
σ* is optimum solubilization ratio;
σ_{avg} is the mean solubilization ratio; and
a⁻ and a⁺ are fitting parameters to match mean solubilization ratio as a function of HLD.

11. The method of claim 5, wherein the symmetrical binodal curve is represented by the mean solubilization ratio at a specific salinity via the following equation:
$${σ}_{avg} = \frac{2 {σ}_{o} {σ}_{w}}{{σ}_{o} + {σ}_{w}}$$

12. The method of claim 1, further comprising determining a number of phases in the microemulsion system.

13. The method of claim 12, further comprising determining composition(s) of the phase(s).

14. The method of claim 12, further comprising:
calculating a mean solubilization ratio σ¹_{avg} assuming a single phase microemulsion system;
calculating the mean solubilization ratio σ_{avg};
determining whether σ¹_{avg} is less than σ_{avg} or σ¹_{avg} is greater than σ_{avg};
concluding that the microemulsion system is a single phase system if σ¹_{avg} is less than σ_{avg}; and
concluding that the microemulsion system is a multiphase system if σ¹_{avg} is greater than σ_{avg}.

15. The method of claim 14, further comprising:
providing an upper HLD limit;
providing a lower HLD limit; and
determining whether a two-phase system exists or a three-phase system exists based on comparing HLD to the lower HLD limit and/or the upper HLD limit.

16. The method of claim 15, further comprising determining whether HLD is less than the lower HLD limit and, if so, concluding a two-phase system exists having a microemulsion phase and an excess oil phase.

17. The method of claim 16, when HLD is less than the lower HLD limit, further comprising using a water solubilization ratio from the overall composition to calculate the oil solubilization ratio from the equation:
$${σ}_{avg} = \frac{2 {σ}_{o} {σ}_{w}}{{σ}_{o} + {σ}_{w}} ;$$
and calculating phase volumes based on the water solubilization ratio and the oil solubilization ratio.

18. The method of claim 15, further comprising determining whether HLD is greater than the lower HLD limit and, if so, concluding a two-phase system exists having a microemulsion phase and an excess brine phase.

19. The method of claim 18, when HLD is greater than the lower HLD limit, further comprising using the oil solubilization ratio from the overall composition to calculate the water solubilization ratio from the equation:
$${σ}_{avg} = \frac{2 {σ}_{o} {σ}_{w}}{{σ}_{o} + {σ}_{w}} ;$$
and calculating phase volumes based on the water solubilization ratio and the oil solubilization ratio.

20. The method of claim 15, further comprising determining whether the lower HLD limit ≤HLD≤the upper HLD limit and, if so, concluding a three-phase system exists having a microemulsion phase, an excess oil phase, and an excess brine phase.

21. The method of claim 20, when the lower HLD limit ≤HLD≤the upper HLD limit, further comprising determining a composition of the three-phase system at an invariant point by: determining an optimum concentration of surfactant (Cₛₘₐₓ) at HLD of 0 with the equation:
$$Csmax = \frac{1}{( 2 {σ}^{*} + 1 )}$$
wherein the concentration of surfactant Cₛ at the invariant point for HLD between the lower HLD limit and zero is determined by linearly interpolating the concentration of surfactant between zero and Cₛₘₐₓ; and
the concentration of surfactant Cₛ at the invariant point for HLD between zero and the upper HLD limit is determined by linearly interpolating the concentration of surfactant between zero and Cₛₘₐₓ; and;
determining the invariant point on the binodal curve represented by the mean solubilization ratio to construct a tie triangle on a ternary diagram using the calculated Cₛ and the equation:
$${σ}_{avg} = \frac{2 {σ}_{o} {σ}_{w}}{{σ}_{o} + {σ}_{w}} ,$$
thus separating a first two-phase system having the microemulsion phase and the excess oil phase and a second two-phase system having the microemulsion phase and the excess brine phase.

22. The method of claim 1, further comprising forecasting field scale oil recovery for the surfactant.

23. The method of claim 1, wherein the optimum salinity S* and the optimum solubilization ratio σ* are determined experimentally.

24. The method of claim 1, wherein the optimum salinity S* and the optimum solubilization ratio σ* are determined by existing predictive correlations.

25. A system for performing a chemical enhanced oil recovery process, comprising:
a processing unit configured to receive a data stream comprising experimental data from the experimental microemulsion system;
a memory communicatively connected to the processing unit, the memory storing instructions which, when executed by the processing unit, cause the system to perform a method for predicting phase behavior in chemical enhanced oil recovery, the method comprising:
determining a hydrophilic-lipophilic difference based on a ratio of salinity to optimum salinity in the microemulsion system containing oil, water, and a surfactant;
determining a mean solubilization ratio as a direct function of the hydrophilic-lipophilic difference at a same state as an optimum solubilization; and
predicting phase behavior based on the determined mean solubilization ratio.

26. A method of performing a chemical enhanced oil recovery process, comprising:
predicting phase behavior of a microemulsion system in an oil reservoir comprising a surfactant, oil, and water from an experimental microemulsion system comprising the same surfactant formulation by:
determining a hydrophilic-lipophilic difference based on a ratio of salinity to optimum salinity in the experimental microemulsion system;
determining a mean solubilization ratio as a direct function of the hydrophilic-lipophilic difference at a same state as an optimum solubilization; and
predicting phase behavior based on the determined mean solubilization ratio.
